(19) United States
(12) Patent Application Publication
(10) Pub. No.: US 2015/0329900 A1
(43) Pub. Date: Nov. 19, 2015

(54) Nucleic Acid Amplification Method

(71) Applicant: QIAGEN GMBH, Hilden (DE)

(72) Inventors: Andy Wende, Burscheid (DE); Sabine Werner, Düsseldorf (DE)

(21) Appl. No.: 14/653,070

(22) PCT Filed: Dec. 17, 2013
(86) PCT No.: PCT/EP2013/076990

§ 371 Date: Jun. 17, 2015

(30) Foreign Application Priority Data
Dec. 18, 2012 (EP) 12197910.8
May 8, 2013 (EP) 13167120.8

(51) Int. Cl. C12Q 1/68

(52) U.S. Cl. CPC C12Q 1/6844

(57) ABSTRACT

The invention relates to a method of performing a helicase dependent amplification (HDA) or thermophilic helicase dependent amplification of a template nucleic acid comprising: (i) combining in a reaction mixture the template nucleic acid; a forward and a reverse HDA primer; a helicase; at least one DNA polymerase and deoxynucleotide triphosphates (dNTPs), (ii) wherein the reaction comprises a linear polyethylene glycol with the formula

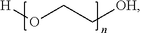

wherein n is between 2 to 50, preferably n is selected from the group comprising 2, 3, 4, 5, 6, 7, 8, 9, 10, 11 and 12. The invention also relates to a kit comprising one or more reagents for performing an HDA reaction, wherein a polyethylene glycol is present in the kit.

### FIELD OF THE INVENTION

The present invention is in the field of molecular biology, particular in the field of nucleic acid amplification and more particular in the field of isothermal nucleic acid amplification.

### BACKGROUND

The polymerase chain reaction (PCR) revolutionized our capabilities to do biological research, and it has been widely used in biomedical research and disease diagnostics. Hand-held diagnostic devices, which can be used to detect pathogens in the field and at point-of-care, are demanded currently. However, the need for power-hungry thermocycling limits PCR application in such a situation. Several isothermal target amplification methods have been developed. Strand-displacement amplification (SDA) combines the ability of a restriction endonuclease to nick the unmodified strand of its target DNA and the action of an exonuclease-deficient DNA polymerase to extend the 3′ end at the nick and displace the downstream DNA strand Transcription-mediated amplification (TMA) uses an RNA polymerase to make RNA from a promoter engineered in the primer region, a reverse transcriptase to produce complementary DNA from the RNA templates and RNase H to remove the RNA from cDNA. In the rolling circle amplification (RCA), a DNA polymerase extends a primer on a circular template, generating tandemly linked copies of the complementary sequence of the template. However, these isothermal nucleic acid amplification methods also have their limitations. Most of them have complicated reaction schemes. In addition, they are incapable of amplifying DNA targets of sufficient length to be useful for many research and diagnostic applications.

In living organisms, a DNA helicase is used to separate two complementary DNA strands during DNA replication. By mimicking nature, a new isothermal DNA amplification technology, helicase-dependent amplification (HDA) was developed. HDA uses a DNA helicase to separate double-stranded DNA (dsDNA) and generates single-stranded templates for primer hybridization and subsequent extension. As the DNA helicase unwinds dsDNA enzymatically, the initial heat denaturation and subsequent thermocycling steps required by PCR can all be omitted. Thus, HDA provides a simple DNA amplification scheme: one temperature from the beginning to the end of the reaction.

Strands of double stranded DNA are first separated by a DNA helicase and coated by single stranded DNA (ssDNA)-binding proteins. In the second step, two sequence specific primers hybridise to each of the complementary strands of the DNA template. A DNA polymerase is then used to extend the primers annealed to the templates to produce a double stranded DNA and the two newly synthesized DNA products are then used as substrates by DNA helicases, entering the next round of the reaction. Thus, a simultaneous chain reaction develops, resulting in exponential amplification of the selected target sequence.

The helicase used in thermophilic HDA (tHDA) belongs to the mismatch repair system in vivo. The E. coli system requires multiple accessory proteins (e.g. at least mutS, mutt and mutH) to generate nicks near the mismatch sites and then load the UvrD, which has high affinity in binding the ends of nucleic acid molecules. However, from a manufacturing perspective, purifying so many accessory proteins to perform in vitro amplifications would be too costly.

Helicases use energy generated by the hydrolysis of nucleoside triphosphates (for example ATP) to break the hydrogen bonds holding the strands together in duplex DNA and RNA. Helicases are involved in every aspect of nucleic acid metabolism in the cell, including DNA replication, repair, recombination, transcription, and protein translation. Helicases can be grouped into two classes based on the mechanism of unwinding: those that translocate in a 5′ to 3′ direction and those that travel in the opposite 3′ to 5′ direction. The 5′ to 3′ helicases usually form hexameric ring structures and are mainly involved in DNA replication.

The UvrD helicase used in HDA reactions is from the class of the 3′ to 5′ translocators. These proteins exist as monomers or dimers and, unlike many other helicases, UvrD helicase is able to melt fully duplex molecules (DNA fragment with blunt ends) and nicked circular DNA molecules. UvrD is involved in the two major DNA repair pathways: methyl-directed mismatch repair and UvrABC-mediated nucleotide excision repair. In the methyl-directed mismatch DNA repair pathway, UvrD is recruited to unwind the DNA strand containing the DNA biosynthetic error.

In tHDA the amplification step takes place at elevated temperatures.

Polyethylene glycols (PEG) have been used to create an artificial molecular crowding condition by excluding water and creating electrostatic interaction with solute polycations (Miyoshi, et al., Biochemistry 41:15017-15024 (2002)). When PEG6000 (7.5%) is added to a DNA ligation reaction, the reaction time is reduced to 5 min (Quick Ligation Kit, New England Biolabs, Inc. (Beverly, Mass.)).

PEG8000 for example is, beside betaine, trehalose, sorbitol, DMSO or BSA, a common and known enhancer for PCR reactions. However in HDA or tHDA reactions the most commonly used PCR enhancers like trehalose, DMSO or sorbitol, do not lead to any improvement of the reaction. Quite the contrary, betaine or PEG8000 do considerably slow down the reaction, respectively inhibit the reaction completely.

Hence it would be helpful to provide additives for the HDA or tHDA reactions, which could improve the reaction.

### DEFINITIONS

The term “nucleic acid” refers to double stranded or single stranded DNA, RNA molecules or DNA/RNA hybrids. Those molecules which are double stranded nucleic acid molecules may be nicked or intact. The double stranded or single stranded nucleic acid molecules may be linear or circular. The duplexes may be blunt ended or have single stranded tails. The single stranded molecules may have secondary structure in the form of hairpins or loops and stems. The nucleic acid may be isolated from a variety of sources including the environment, food, agriculture, fermentations, biological fluids such as blood, milk, cerebrospinal fluid, sputum, saliva, stool, lung aspirates, swabs of mucosal tissues or tissue samples or cells. Nucleic acid samples may obtained from cells or viruses and may include any of: chromosomal DNA, extra chromosomal DNA including plasmid DNA, recombinant DNA, DNA fragments, messenger RNA, transfer RNA, ribosomal RNA, double stranded RNA or other RNAs that occur in cells or viruses. The nucleic acid may be isolated, cloned or synthesized in vitro by means of chemical synthesis. Any of the above described nucleic acids may be subject to modification where individual nucleotides within the nucleic acid are chemically altered (for example, by methylation). Modifications may arise naturally or by in vitro synthesis. The term “duplex” refers to a nucleic acid molecule that is double stranded in whole or part.

The term “target” or “template” nucleic acid refers to a whole or part of nucleic acid to be selectively amplified and which is defined by 3′ and 5′ boundaries. The target nucleic acid may also be referred to as a fragment or sequence that is intended to be amplified. The size of the target nucleic acid to be amplified may be, for example, in the range of about or at least 50 to 1000, 50 to 500, 50 to 250, 75 to 150 bases or kilobases. The target nucleic acid may be contained within a longer double stranded or single stranded nucleic acid. Alternatively, the target nucleic acid may be an entire double stranded or single stranded nucleic acid. The template can also be modified nucleic acid, e.g. by organic groups such as methyl groups, biotin, formaldehyde modified nucleic acids, and such.

If RNA is used as a template, reverse transcription into cDNA have to be performed prior to initiation HDA. Synthesis of cDNA may be performed prior to HDA in a different reaction and/or different reaction milieu (two-step process) or can be performed within the HDA reagents (one-step process). The target nucleic acid may be damaged and may repaired prior amplification (e.g. repair of abasic sites). The target nucleic acid may have no primer binding site. In this case the missing primer binding site may be attached e.g. by ligation so that HDA can be performed.

The terms “melting,” “unwinding” or “denaturing” refer to separating all or part of two complementary strands of a nucleic acid duplex.

The term of “hybridization” refers to binding of an oligonucleotide primer to a region of the single-stranded nucleic acid template under the conditions in which a primer binds only specifically to its complementary sequence on one of the template strands, not other regions in the template. The specificity of hybridization may be influenced by inter alia, the length of the oligonucleotide primer, the temperature in which the hybridization reaction is performed, the ionic strength, GC content and the pH.

The term “primer” refers to a single stranded nucleic acid capable of binding to a single stranded region on a target nucleic acid to facilitate polymerase dependent replication of the target nucleic acid. The invention envisages the use of a forward and a reverse primer. Preferably, the primers described herein do not or are not predicted to form secondary structures, complete or partial hairpins, in any given phase of an HDA reaction (e.g., during melting, hybridization/annealing and/or extension).

Generally, primer pairs suitable for use in HDA are short synthetic oligonucleotides, for example, having a length of exactly, about or at least 8, 9, 10, 11, 12, 13, 14, 15, 16, 17, 18, 19, 20, 22, 23, 24, 25, 26, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more nucleotide bases. Preferably the primers are between about 5 and 60, 10 and 50, 15 and 30 nucleotide bases.

Oligonucleotide primer design involves various parameters such as string-based alignment scores, melting temperature, primer length and GC content (Kampke et al., Bioinformatics 17:214-225 (2003)). When designing a primer, one of the important factors is to choose a sequence within the target fragment which is specific to the nucleic acid molecule to be amplified. The other important factor is to decide the melting temperature of a primer for HDA reaction. The melting temperature of a primer is determined by the length and GC content of that oligonucleotide. Preferably the melting temperature of a primer is exactly, about or at least 3, 4, 5, 6, 7, 8, 9, 10, 11, 12, 13, 14, 15, 16, 17, 18, 19, 20, 22, 23, 24, 25, 26, 26, 27, 28, 29, 30, 31, 32, 33, 34, 35, 36, 37, 38, 39, 40, 41, 42, 43, 44, 45, 46, 47, 48, 49, or 50 degrees Celsius above or below the temperature at which the hybridization and amplification will take place. More preferably, the melting temperature is about 10 degrees Celsius below the hybridization or amplification temperature of the HDA to 30 degrees Celsius above the hybridization or amplification temperature of the HDA, or 15 degrees Celsius below the hybridization or amplification temperature of the HDA to 25 degrees Celsius above than the temperature at which the hybridization and amplification will take place. For example, if the temperature of the hybridization and amplification is set at 37 degrees Celsius when using the E. coli UvrD helicase preparation, the melting temperature of a pair of primers designed for this reaction should be in a range between about 27 degrees Celsius to about 67 degrees Celsius.

In certain embodiments, when the temperature of the hybridization and amplification is 60 degree Celsius, the melting temperature of a pair of primers designed for that reaction should be in a range between 45 and 90 degrees Celsius. To choose the best primer for a HDA reaction, a set of primers with various melting temperatures can be tested in parallel assays. More information regarding primer design is described by Kampke et al., Bioinformatics 17:214-225 (2003).

The term “cofactor” refers to small-molecule agents that are required for the helicase unwinding activity. Helicase cofactors include nucleoside triphosphate (NTP) and deoxy-nucleoside triphosphate (dNTP) and magnesium (or other divalent cations). For example, ATP (adenosine triphosphate) may be used as a cofactor for UvrD helicase at a concentration in the range of 0.1-100 mM and preferably in the range of 1 to 10 mM (for example 3 mM). Similarly, dTTP (deoxythymidine triphosphate) may be used as a cofactor for T7 Gp4B helicase in the range of 1-10 mM (for example 3 mM).

The term “helicase” refers here to any enzyme capable of unwinding a double stranded nucleic acid enzymatically. For example, helicases are enzymes that are found in all organisms and in all processes that involve nucleic acid such as replication, recombination, repair, transcription, translation and RNA splicing. (Kornberg and Baker, DNA Replication, W.H. Freeman and Company (2nd ed. (1992)), especially chapter 11). Any helicase that translocates along DNA or RNA in a 5′ to 3′ direction or in the opposite 3′ to 5′ direction may be used in present embodiments of the invention. This includes helicases obtained from prokaryotes, viruses, archaea, and eukaryotes or recombinant forms of naturally occurring enzymes as well as analogues or derivatives having the specified activity. Examples of naturally occurring DNA helicases, described by Kornberg and Baker in chapter 11 of their book, DNA Replication, W.H. Freeman and Company (2nd ed. (1992)), include E. coli helicase I, II, III, & IV, Rep, DnaB, PriA, PcrA, T4 Gp41 helicase, T4 Dda helicase, T7 Gp4 helicases, SV40 Large T antigen, yeast RAD. Additional helicases that may be useful in HDA include RecQ helicase (Harmon and Kowalczykowski, J. Biol. Chem. 276:232-243 (2001)), thermostable UvrD helicases from T. tengcongensis and T. thermophilus (Collins and McCarthy, Extremophiles. 7:35-41. (2003)), thermostable DnaB helicase from T. aquaticus (Kaplan and Steitz, J. Biol. Chem. 274:6889-6897 (1999)), and MCM helicase from archaeal and eukaryotic organisms ((Grainge et al., Nucleic Acids Res. 31:4888-4898 (2003)).

Non-limiting examples of helicases for use in present embodiments may also be found at the following web address: http://blocks.fhcrc.org (Get Blocks by Keyword: helicase). This site lists 49 Herpes helicases, 224 DnaB helicases, 250 UvrD-helicases and UvrD/Rep helicases, 276 DEAH_ATP-dependent helicases, 147 Papillom_E1 Papillomavirus helicase E1 protein, 608 Viral helicasel Viral (superfamily 1) RNA helicases and 556 DEAD_ATP-dependent helicases. Examples of helicases that generally replicate in a 5′ to 3′ direction are T7 Gp4 helicase, DnaB helicase and Rho helicase, while examples of helicases that replicate in the 3′-5′ direction include UvrD helicase, PcrA, Rep, NS3 RNA helicase of HCV.

Originally, HDA was described by using UvrD like helicases from different organisms like E. coli or Thermoanaerobacter tengcongensis. Other helicase may be of equal functionality in HDA e.g PcrA (Staphylococcus), RecD or Rep from E. coli, Dda (T4-phage), or others.

The helicase can be provided in a “helicase preparation.” The helicase preparation refers to a mixture of reagents which when combined with a DNA polymerase, a nucleic acid template, four deoxynucleotide triphosphates, and primers are capable of achieving isothermal, exponential and specific nucleic acid amplification in vitro.

More particularly, the helicase preparation includes a helicase, an energy source such as a nucleotide triphosphate (NTP) or deoxynucleotide triphosphate (dNTP), and a single strand DNA binding protein (SSB). One or more additional reagents may be included in the helicase preparation, where these are selected from the following: one or more additional helicases, an accessory protein, small molecules, chemical reagents and a buffer.

Where a thermostable helicase is utilized in a helicase preparation, the presence of a single stranded binding protein is optional.

The term “HDA system”, “tHDA system”, “HDA kit” or “tHDA kit” is used herein to describe a group of interacting elements for performing the function of amplifying nucleic acids according to the Helicase-Dependent Amplification method described herein. The HDA system includes HDA reagents such as a forward and reverse primer, a helicase preparation, a polymerase and optionally a topoisomerase.

HDA reagents can also include DNA binding proteins (e.g. SSB, MutS, MutL or others), BSA, pyrophosphatase, kinases, other polymerases, a reverse transcriptase (to convert RNA template to DNA prior to HDA amplification), sugars, sugar alcohols, polymers (e.g. PEG), dextrose, polymers from natural source (e.g. from algae, plants, fungi), enzymes to repair target nucleic acid prior amplification, cofactors and/or accessory proteins.

The HDA reagents may also include uracil-N-glycosylase (UNG), a DNA repair enzyme that hydrolyzes the base-ribose bond at uracil residues, can be used to eliminate DNA contamination from previously amplified PCR products. UNG treatment prevents replication of uracil-containing DNA by causing the DNA polymerase to stall at the resulting abasic sites. For UNG to be effective against contamination, the products of previous amplifications may be synthesized in the presence of dUTP. This is may be accomplished by substituting dUTP for some or all of the dTTP in the reaction.

For example, the UvrD HDA system may be constituted by mixing together, a UvrD helicase preparation (for example, an E. coli UvrD helicase preparation or a Tte-UvrD helicase preparation) and a DNA polymerase such as Exo-Klenow Fragment, DNA polymerase Large fragment, Exo+ Klenow Fragment or T7 Sequenase.

Another example is the T7 HDA system which includes a T7 helicase preparation (T7 Gp4B helicase, T7 Gp2.5 SSB, and dTTP), and T7 Sequenase.

Another example is RecBCD HDA system which includes a RecBCD preparation (RecBCD helicase with T4gp 32) and T7 Sequenase.

Any selected HDA system may be optimized by substitution, addition, or subtraction of elements within the mixture as discussed in more detail below.

Helicases show improved activity in the presence of single-strand binding proteins (SSB). In these circumstances, the choice of SSB is generally not limited to a specific protein. Examples of single strand binding proteins are T4 gene 32 protein, E. coli SSB, T7 gp2.5 SSB, phage phi29 SSB (Kornberg and Baker, supra (1992)) and truncated forms of the aforementioned. Topoisomerase can be used in long HDA reactions to increase the ability of HDA to amplify long target amplicons. When a very long linear DNA duplex is separated by a helicase, the swivel (relaxing) function of a topoisomerase removes the twist and prevents over-winding (Kornberg and Baker, supra (1992)). For example, E. coli topoisomerase I (Fermentas, Vilnius, Lithuania) can be used to relax negatively supercoiled DNA by introducing a nick into one DNA strand. In contrast, E. Coli DNA gyrase (topoisomerase II) introduces a transient double-stranded break into DNA allowing DNA strands to pass through one another (Kornberg and Baker, supra (1992)). Amplified nucleic acid product may be detected by various methods including ethidium-bromide staining and detecting the amplified sequence by means of a label selected from the group consisting of a radiolabel, a fluorescent-label, and an enzyme. For example HDA amplified products can be detected in real-time using fluorescent-labeled LUX™ Primers (Invitrogen Corporation, Carlsbad, Calif.) which are oligonucleotides designed with a fluorophore close to the 3′ end in a hairpin structure. This configuration intrinsically renders fluorescence quenching capability without separate quenching moiety. When the primer becomes incorporated into double-stranded amplification product, the fluorophore is dequenched, resulting in a significant increase in fluorescent signal.

Although other isothermal nucleic acid amplification methods such as Strand-Displacement Amplification can amplify target at a constant temperature without thermo-cycling, they do require an initial denaturation step to generate single-stranded template. An advantage of embodiments of the method described herein is that both unwinding by helicase and amplification can effectively occur at a single temperature throughout. Alternatively, the temperature is raised to assist initial unwinding of the target nucleic acid by the helicase and the amplification then proceeds at a single temperature.

HDA can be used in place of PCR for amplification of reverse transcribed product of RNA. In addition, HDA is useful for quantitative amplification such as found to be useful in gene expression studies and environmental analyses. Accordingly, where it is desirable to determine the amounts of a target nucleic acid, HDA can be utilized in a real time end point assay. Accordingly, HDA may be used to determine the relative amounts of messenger RNA in a cell in gene expression studies. For example, calibrated gene expression profiles described in WO 01/25473 can be generated using quantitative helicase dependent amplification or Q-HDA.

Real time HDA may be used as a sensitive technique to determine amounts of an organism in a contaminated sample such as E. coli in seawater. Real time detection uses sensitive markers such as fluorescence in a HDA reaction.

HDA may be used in the context of a compact device for use in field activities and/or laboratory diagnoses. For example, HDA may be practiced in a microfluidic environment. Microfluidics technologies (lab on a chip) are rapidly emerging as key strategies for cost and time saving by performing biochemical analyses in miniaturized environments usually at nanoliter scale. Microfluidics technologies have great potential to be used as field-portable equipment in pathogen detection when combining with a nucleic acid amplification and detection method. The ability of HDA to amplify nucleic acids in an isothermal condition without initial heat-denaturation makes it a good candidate for the nucleic acid amplification process in a microfluidic device. Similarly, HDA may be used either in kits or in laboratory amplification procedures to create response profiles of the sort described in International Publication No. WO 02/02740 or for monitoring disease (U.S. Publication No. 2001018182).

HDA may be used for amplifying target nucleic acid from different sources and having different sequences. For example, longer target sequence (>2 kb) can be amplified by the T7 Gp4B-based HDA system. The method of using Helicase-Dependent Amplification to amplify nucleic acids can be performed using different helicase preparations, such as a helicase preparation containing T7 Gp4B helicase, or a helicase preparation containing more than one helicase, such as T7 Gp4B helicase and UvrD helicase.

The ability of HDA to amplify of as little as 10 copies of bacterial genomic DNA supports the use of HDA for molecular diagnostics application of infectious diseases caused by pathogenic bacteria, for example Chlamydia trachomatis and Neisseria gonorrhoeae. The demonstration that target sequences can be amplified from human genomic DNA samples supports the use of HDA in identifying genetic alleles corresponding to a particular disease including single nucleotide polymorphisms and forensic applications that rely on characterizing small amounts of nucleic acid at the scene of a crime or at an archeological site.

“Isothermal amplification” refers to amplification which occurs at a single temperature. This does not include the single brief time period (less than 15 minutes) at the initiation of amplification which may be conducted at the same temperature as the amplification procedure or at a higher temperature. Depending on the source of enzymes that are used for HDA, the reaction can be performed at low temperatures (<50° C.) e.g., at least or about 30, 31, 32, 33, 34, 35, 36, 37, 38, 39, 40, 41, 42, 43, 44, 45, 46, 47, 48, 49 degrees Celsius; or at high temperatures (≧50° C.), e.g., at least or about 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115 or more degrees Celsius.

As used herein, the term “dNTP” refers to deoxyribonucleoside triphosphates. Non-limiting examples of such dNTPs are dATP, dGTP, dCTP, dTTP, dUTP, which may also be present in the form of labelled derivatives, for instance comprising a fluorescence label, a radioactive label, a biotin label. dNTPs with modified nucleotide bases are also encompassed, wherein the nucleotide bases are for example hypoxanthine, xanthine, 7-methylguanine, inosine, xanthinosine, 7-methylguanosine, 5,6-dihydrouracil, 5-methylcytosine, pseudouridine, dihydrouridine, 5-methylcytidine. Furthermore, ddNTPS of the above-described molecules are encompassed in the present invention.

### DETAILED DESCRIPTION OF THE INVENTION

Nucleic acid samples to be used in tHDA reactions are usually purified using a chaotropic salt/silica method. In these methods ethanol is usually used in purification buffers. Remnants of ethanol in the test sample might influence the performance of the tHDA reaction. Hence the use of alternatives is preferred. A potential alternative might be polyethylene glycol.

Polyethylene glycols are polyethers with the general formula:

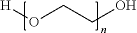

Polyethylene glycol as used in this invention refers to polyethylene glycols wherein n is between 2 and 50. In a preferred embodiment the polyethylene glycol is linear and not branched.

The inventors found unexpectedly, that the presence of a linear polyethylene glycol (PEG) with a short chain length (n between 2 to 50), especially tetraethylene glycol (n=4, TEG), not only does not have any negative effects on the tHDA reaction, but surprisingly improves the reaction speed. Hence the invention relates to the use of polyethylene glycols, preferably tetraethylene glycol, as additives in helicase dependent amplification reactions or preferably as additives in thermophilic helicase dependent amplification reactions.

The present invention further relates to a method of performing a helicase dependent amplification (HDA) or preferably a thermophilic helicase dependent amplification (tHDA) of a template nucleic acid comprising: combining in a reaction mixture the template nucleic acid; a forward and a reverse HDA primer; a helicase; and deoxynucleoside triphosphates (dNTPs), wherein the reaction comprises a linear polyethylene glycol with the formula:

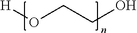

and n is between 2 and 50, preferably between 2 and 25, more preferably between 2 and 15.

In even more preferred embodiments of the invention n is selected from the group comprising 2 (diethylene glycol), 3 (triethylene glycol), 4 (tetratethylene glycol), 5 (pentaethylene glycol), 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 and 40.

In the most preferred embodiment of the invention n is selected from the group comprising 2, 3, 4, 5, 6, 7, 8, 9, 10, 11 and 12.

The inventors found, that the use of polyethylene glycol even in higher concentrations did not have any negative effect on tHDA reactions and observed an improvement of the amplification speed at concentrations of 2% to 5% volume percent of polyethylene glycol in the reaction mixture. Hence the invention relates to a method of performing HDA reactions or tHDA reactions, wherein the polyethylene glycol is present at between 0.1% and 20% (volume percent), preferably at between 2% to 8% (volume percent), even more preferably 2% to 4% (volume percent). In the most preferred embodiments the concentration of the PEG is 5% (volume percent) or lower.

The polyethylene glycol may be modified.

One modification is poly(ethylene glycol) dimethyl ether.

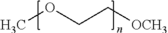

PEG may also be modified as a monomethyl ether. In one embodiment one or both ends have an alkan cap with 1-10 C's.

PEG may also be modified with a mono- or diphenyl ether. PEG may herein also be a polypropylene glycol with or without the modifications just mentioned.

For helicase dependent amplifications a variety of helicases can be used. Hence the invention relates to performing an HDA or tHDA amplification, wherein the helicase in the reaction is selected from the group of T7 Gp4 helicase, DnaB helicase, Rho helicase, UvrD helicase, PcrA, Rep and NS3 RNA helicase.

In a preferred embodiment of the invention the helicase is UvrD helicase. In an even more preferred embodiment the helicase is the UvrD-like helicase of T. tengcongensis.

The present invention is not limited to singleplex HDA or tHDA. Hence in one embodiment of the invention the HDA or tHDA reaction is a singleplex HDA or tHDA. In another embodiment of the invention the HDA or tHDA reaction is a multiplex HDA or tHDA.

The inventors tried to improve HDA or tHDA reactions, to circumvent problems, which might occur by the use of ethanol during nucleic acid purification. Nucleic acid purification is usually carried out, using a chaotropic salt/silica method, wherein the purification buffer usually contains ethanol. Hence the invention relates to a method for HDA or tHDA, wherein the nucleic acid was prepared using a chaotropic salt/silica method, wherein the purification buffer does not contain ethanol.

In a preferred embodiment of the Invention the nucleic acid sample is prepared using a chaotropic salt/silica method, wherein the purification buffer comprises a linear polyethylene glycol with the formula

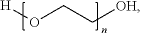

wherein n is between 2-50, preferably between 2-25, more preferably between 2-15, most preferably n is selected from the group comprising 2, 3, 4, 5, 6, 7, 8, 9, 10, 11 and 12, wherein the concentration of the PEG is 5% (volume percent) or lower.

In addition the invention relates to a method for the generation of a kit for performing an HDA or tHDA reaction comprising the steps of (a) providing a helicase and a DNA polymerase; (b) optionally providing a buffer, in which both the helicase and DNA polymerase show activity; (c) providing a linear polyethylene glycol with the formula

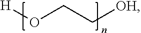

wherein n is between 2-50, preferably between 2-20, more preferably between 2-15, most preferably n is selected from the group comprising 2, 3, 4, 5, 6, 7, 8, 9, 10, 11 and 12, in such a way, that the polyethylene glycol is present 5% (volume percent) or lower, or preferably present at between 2% and 5% (volume percent), or more preferably at between 2% and 4% (volume percent) in the final reaction volume; (d) optionally providing primers for a HDA or tHDA reaction; (e) optionally providing deoxynucleotide triphosphates; (f) alternatively providing a buffer, which comprises polyethylene glycol and optionally primers and optionally deoxynucleotide triphosphates (master mix) (g) combining the provided components in a kit, each in separate form, optionally including instructions, ready for distribution.

The Invention further relates to a kit comprising one or more reagents for performing an HDA or tHDA reaction, wherein a linear polyethylene glycol, preferably tetraethylene glycol is present in the kit.

In a preferred embodiment of the present invention the kit comprises a helicase in an active or inactive form, a DNA polymerase in an active or inactive form and a polyethylene glycol, preferably tetraethylene glycol.

In a more preferred embodiment of the invention the kit additionally comprises a buffer, deoxynucleotide triphosphates and a polyethylene glycol.

In a more preferred embodiment of the invention the kit additionally comprises at least one or more forward HDA or tHDA primer and/or at least one or more reverse HDA or tHDA primer.

In an even more preferred embodiment of the invention the linear polyethylene glycol in the kit has the formula

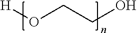

and n is between 2-50, preferably 2-25, more preferably 2-15.

In yet a more preferred embodiment of the invention n of the polyethylene glycol is selected from the group comprising 2, 3, 4, 5, 6, 7, 8, 9, 10, 11 and 12.

In the most preferred embodiment of the invention the kit additionally comprises one or more reagents for nucleic acid sample preparation, using a chaotropic salt/silica method, wherein the purification buffer comprises a linear polyethylene glycol with the formula:

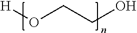

and n is between 2-50, preferably between 2-25, more preferably between 2-15, and most preferably n selected from the group comprising 2, 3, 4, 5, 6, 7, 8, 9, 10, 11 and 12, and wherein the concentration of the PEG is 5% (volume percent) or lower.

The present invention might be used in diagnostic assays, e.g. comprising primer/probe systems. The invention might be used to detect specific nucleic acid sequences in a purified nucleic acid sample. In one embodiment of the invention the nucleic acid sample was purified using a chaotropic salt/silica method, preferably, wherein the purification buffer did not contain ethanol, even more preferably, wherein the purification buffer comprises a linear polyethylene glycol with the formula:

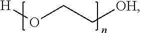

wherein n is between 2 to 50, preferably between 2 to 25, more preferably between 2 to 15 and most preferred n is selected from the group comprising 2, 3, 4, 5, 6, 7, 8, 9, 10, 11 and 12 and wherein the concentration of the PEG is 5% (volume percent) or lower.

The detection of the HDA or tHDA amplicon could be done using fluorescence/quencher labelled probes, which hybridize to the amplicon and subsequently fluorescence can be detected.

Surprisingly the inventors found, that the addition of polyethylene glycol, preferably tetraethylene glycol, resulted in an improved amplification speed, leading to a more rapid detection of fluorescence.

### EXAMPLES

### Example 1

### Effect of Ethanol and Tetraethylene Glycol on tHDA Reactions

In these experiments the influence of tetraethylene glycol on tHDA reaction was analyzed and compared with the influence of ethanol on the reaction under the otherwise same conditions. The components used had the following concentrations in the final reaction volume:

160 nM forward primer (specific for N. gon.)
480 nM reverse primer (specific for N. gon.)
60 nM probe (specific for N. gon)
100 cp N. gonorrhoeae genomic DNA
0-5% (v/v) ethanol/TEG

### 40 mM NaCl, 10 mM KCl, 5 mM MgSO₄

20 mM Tris/HCl pH 8.8, 10% trehalose
0.5 mM dNTP mix, 5 mM dATP
400 ng Tte Helicase, 75 ng Et SSB, 40 U Gst Pol. LF, 2 U Gst Pol. FL (all obtained from Biohelix)

The components were put in two different mixtures. Mix 1 (primer, template, test substances) and mix 2 (other substances) were put on ice. 15 μL aliquots of mix 2 were aliquoted in PCR tubes and incubated for 1 min at 63° C. in a PCR cycler (Biorad CFX96). The tHDA reaction was started subsequently by the addition of 10 μL of Mix 1 and mixing. The fluorescence was measured over 40 min in 1 min intervals. All measurements were performed in duplicate.

FIG. 2 and table 1 show the results of the experiment in the presence of 0-5% ethanol. The so called “Ct-value” corresponds to the incubation time, after which the measured fluorescence of an amplification reaction exceeds a defined threshold.

TABLE 1
Time (in minutes) until the measured fluorescence
of the HDA reactions with 0-5% EtOH in the reaction
mixture exceeds a defined threshold.

| | EtOH (v/v) | | | | | | | |
|---|---|---|---|---|---|---|---|---|
| | 0 | 0.1 | 0.2 | 0.5 | 1 | 2 | 3 | 5 |
| average | 18.74 | 20.20 | 22.41 | 18.82 | 19.69 | 22.7 | 31.37 | 39.45 |
| Ct-value | | | | | | | | |

The data shows that small amounts of ethanol (up to 1%) might lead to slight fluctuations of the Ct-value, but does not heavily interfere with the HDA reaction (the double value at 0.2% EtOH is considered to be an outlier). EtOH concentrations of 2% and higher however, did heavily influence the HDA reaction. FIG. 1 shows that, besides an increased Ct-value, the final fluorescence intensity of the reactions is strongly reduced.

FIG. 3 and table 2 summarize the results of the experiments in case of the presence of 0-5% TEG in the reaction mixture.

TABLE 2
Time (in minutes) until the measured fluorescence
of the HDA reactions with 0-5% TEG in the reaction
mixture exceeds a defined threshold

| | TEG (v/v) | | | | | | | |
|---|---|---|---|---|---|---|---|---|
| | 0 | 0.1 | 0.2 | 0.5 | 1 | 2 | 3 | 5 |
| average | 18.36 | 18.70 | 19.36 | 18.15 | 18.12 | 16.29 | 15.44 | 16.40 |
| Ct-value | | | | | | | | |

The data show that TEG does not have any negative effect on the HDA reaction, even up to the used maximum of 5% in the reaction mixture. On the contrary we found, that the Ct-value decreases at TEG concentrations of 2% or higher. This suggests that the reaction is more efficient under these conditions. FIG. 2 also shows that the fluorescence progress is also not negatively affected by the addition of TEG.

### Example 2

### Effect of Tetraethylene Glycol on tHDA Reactions—Amplification of N. gonorrhoeae DNA

In these experiments the Influence of TEG on the amplification of N. gonorrhoeae DNA was tested.

The components used had the following concentrations in the final reaction volume:

160 nM forward primer (specific for N. gonorrhoeae)
480 nM reverse primer (specific for N. gonorrhoeae)
60 nM probe (specific for N. gonorrhoeae)
100 cp N. gonorrhoeae genomic DNA
0-5% (v/v) TEG

### 40 mM NaCl, 10 mM KCl, 5 mM MgSO₄

### 20 mM Tris/HCl pH 8.8

0.5 mM dNTP mix, 5 mM dATP
400 ng Tte Helicase, 75 ng Et SSB, 40 U Gst Pol. LF, 2 U Gst Pol. FL (all obtained from Biohelix)

The components were put in two different mixtures. Mix 1 (primer, template, test substances) and mix 2 (other substances) were put on ice. 15 μL aliquots of mix 2 were aliquoted in PCR tubes and incubated for 1 min. at 63° C. in a PCR cycler (Biorad CFX96). The tHDA reaction was started subsequently by the addition of 10 μl of Mix 1 and mixing. The fluorescence was measured over 40 min in 1 min intervals. All measurements were performed in duplicate.

TABLE 3
Average Ct- and fluorescence values of tHDA reactions with primer/probe combinations
for the detection of N. gonorrhoeae in the presence of 0 to 5% TEG.

| | TEG (v/v) | | | | | | |
|---|---|---|---|---|---|---|---|
| | 0 | 0.5 | 1 | 2 | 3 | 4 | 5 |
| Ct-average | 12.9 | 12.97 | 13.83 | 11.76 | 13.90 | 12.59 | n.a. |
| Final fluorescence | 1219 | 1194 | 1218 | 1432 | 1071 | 1547 | n.a. |

### Example 3

### Effect of Tetraethylene Glycol on tHDA Reactions—Amplification of C. trachomatis DNA

The components used had the following concentrations in the final reaction volume:

160 nM forward primer (specific for C. trachomatis)
480 nM reverse primer (specific for C. trachomatis)
60 nM probe (specific for C. trachomatis)
100 cp C. trachomatis genomic DNA
0-5% (v/v) TEG

### 40 mM NaCl, 10 mM KCl, 5 mM MgSO₄

### 20 mM Tris/HCl pH 8.8

0.5 mM dNTP mix, 5 mM dATP
400 ng Tte Helicase, 75 ng Et SSB, 40 U Gst Pol. LF, 2 U Gst Pol. FL (all obtained from Biohelix)

The components were put in two different mixtures. Mix 1 (primer, template, test substances) and mix 2 (other substances) were put on ice. 15 μL aliquots of mix 2 were aliquoted in PCR tubes and incubated for 1 min. at 63° C. in a PCR cycler (Biorad CFX96). The tHDA reaction was started subsequently by the addition of 10 μL of Mix 1 and mixing. The fluorescence was measured over 40 min in 1 min intervals. All measurements were performed in duplicate.

TABLE 4
Average Ct- and fluorescence values of tHDA reactions with primer/probe combinations
for the detection of C. trachomatis in the presence of 0 to 5% TEG.

| | TEG (v/v) | | | | | | |
|---|---|---|---|---|---|---|---|
| | 0 | 0.5 | 1 | 2 | 3 | 4 | 5 |
| Ct-average | 12.02 | 11.39 | 11.07 | 10.93 | 12.19 | 11.94 | 12.66 |
| Final fluorescence | 118 | 186 | 246 | 188 | 169 | 187 | 163 |

### Example 4

### Effect of Tetraethylene Glycol on tHDA Reactions—Amplification of Human DNA

The components used had the following concentrations in the final reaction volume:

160 nM forward primer (specific for human DNA)
480 nM reverse primer (specific for human DNA)
60 nM probe (specific for human DNA)
100 cp human genomic DNA
0-5% (v/v) TEG

### 40 mM NaCl, 10 mM KCl, 5 mM MgSO₄

### 20 mM Tris/HCl pH 8.8

0.5 mM dNTP mix, 5 mM dATP
400 ng Tte Helicase, 75 ng Et SSB, 40 U Gst Pol. LF, 2 U Gst Pol. FL (all obtained from Biohelix)

The components were put in two different mixtures. Mix 1 (primer, template, test substances) and mix 2 (other substances) were put on ice. 154 aliquots of mix 2 were aliquoted in PCR tubes and incubated for 1 min. at 63° C. in a PCR cycler (Biorad CFX96). The tHDA reaction was started subsequently by the addition of 10 μL of Mix 1 and mixing. The fluorescence was measured over 40 min in 1 min intervals. All measurements were performed in duplicate.

TABLE 5
Average Ct- and fluorescence values of tHDA reactions with primer/probe combinations
for the detection of human DNA in the presence of 0 to 5% TEG.

| | TEG (v/v) | | | | | | |
|---|---|---|---|---|---|---|---|
| | 0 | 0.5 | 1 | 2 | 3 | 4 | 5 |
| Ct-average | 14.15 | 13.14 | 12.20 | 12.19 | 13.73 | 13.06 | 12.49 |
| Final fluorescence | 304 | 371 | 408 | 405 | 425 | 527 | 886 |

### Example 5

### Effect of Tetraethylene Glycol on tHDA Reactions—Amplification of M. tuberculosis DNA

The components used had the following concentrations in the final reaction volume:

160 nM forward primer (specific for M. tuberculosis)
480 nM reverse primer (specific for M. tuberculosis)
60 nM probe (specific for M. tuberculosis)
100 cp M. tuberculosis genomic DNA
0-5% (v/v) TEG

### 40 mM NaCl, 10 mM KCl, 5 mM MgSO₄

### 20 mM Tris/HCl pH 8.8

0.5 mM dNTP mix, 5 mM dATP
400 ng Tte Helicase, 75 ng Et SSB, 40 U Gst Pol. LF, 2 U Gst Pol. FL (all obtained from Biohelix)

The components were put in two different mixtures. Mix 1 (primer, template, test substances) and mix 2 (other substances) were put on ice. 15 μL aliquots of mix 2 were aliquoted in PCR tubes and incubated for 1 min. at 63° C. in a PCR cycler (Biorad CFX96). The tHDA reaction was started subsequently by the addition of 10 μL of Mix 1 and mixing. The fluorescence was measured over 40 min in 1 min intervals. All measurements were performed in duplicate.

TABLE 6
Average Ct values of tHDA reactions with primer/probe
combinations for the detection of M. tuberculosis
in the presence of 0 to 5% TEG.

| | TEG (v/v) | | | | | | |
|---|---|---|---|---|---|---|---|
| | 0 | 0.5 | 1 | 2 | 3 | 4 | 5 |
| Ct- | 16.11 | 17.04 | 16.05 | 14.27 | 13.05 | 12.06 | 13.18 |
| average | | | | | | | |

The following examples show that polyethylene glycols with chain length of 2-12 ethylene monomers show the same surprising effect on the tHDA amplification speed.

### Example 6

### Effect of Different Ethylene Glycols on tHDA Reactions—Amplification of N. Gonorrhoeae DNA

The components used had the following concentrations in the final reaction volume:

160 nM forward primer (specific for N. gonorrhoeae)
480 nM reverse primer (specific for N. gonorrhoeae)
60 nM probe (specific for N. gonorrhoeae)
100 cp N. gonorrhoeae genomic DNA
2% (v/v) Di-, Tri-, Tetra-, Penta-, or Hexa-Ethylene glycol

### 40 mM NaCl, 10 mM KCl, 5 mM MgSO₄

### 20 mM Tris/HCl pH 8.8

0.5 mM dNTP mix, 5 mM dATP
400 ng Tte Helicase, 75 ng Et SSB, 80 U modified Gst Pol. LF (all obtained from Biohelix)

The components were pipetted into the reaction tube on ice and tubes have been mixed well. The tHDA reaction was started by putting the reaction tubes into the pre-heated PCR cycler (Biorad CFX 96). The fluorescence was measured over 40 min in 1 min intervals. All measurements were performed in duplicate.

TABLE 7
Average Ct values of tHDA reactions with primer/probe
combinations for the detection of N. gonorrhoeae in
the presence of 2% 2-, 3-, 4-, 5-, 6-Ethylene glycol.

| chain length of Ethylene glycol | 2 | 3 | 4 | 5 | 6 |
|---|---|---|---|---|---|
| Ct-average | 20.28 | 18.22 | 17.26 | 16.53 | 14.55 |

### Example 7

### Effect of Different Ethylene Glycols on tHDA Reactions—Amplification of C. Trachomatis DNA

The components used had the following concentrations in the final reaction volume:

160 nM forward primer (specific for C. trachomatis)
480 nM reverse primer (specific for C. trachomatis)
60 nM probe (specific for C. trachomatis)
100 cp C. trachomatis genomic DNA
2% (v/v) Di-, Tri-, Tetra-, Penta-, or Hexa-Ethylene glycol

### 40 mM NaCl, 10 mM KCl, 5 mM MgSO₄

### 20 mM Tris/HCl pH 8.8

0.5 mM dNTP mix, 5 mM dATP
400 ng Tte Helicase, 75 ng Et SSB, 80 U modified Gst Pol. LF (all obtained from Biohelix)

The components were pipetted into the reaction tube on ice and tubes have been mixed well. The tHDA reaction was started by putting the reaction tubes into the pre-heated PCR cycler (Biorad CFX 96). The fluorescence was measured over 40 min in 1 min intervals. All measurements were performed in duplicate.

TABLE 8
Average Ct values of tHDA reactions with primer/probe
combinations for the detection of C. trachomatis in
the presence of 2% 2-, 3-, 4-, 5-, 6-Ethylene glycol.

| chain length of Ethylene glycol | 2 | 3 | 4 | 5 | 6 |
|---|---|---|---|---|---|
| Ct-average | 13.6 | 13.04 | 15.1 | 13.03 | 13.33 |

### Example 8

### Effect of Different Ethylene Glycols on tHDA Reactions—Amplification of C. Trachomatis DNA

The components used had the following concentrations in the final reaction volume:

160 nM forward primer (specific for C. trachomatis)
480 nM reverse primer (specific for C. trachomatis)
60 nM probe (specific for C. trachomatis)
100 cp C. trachomatis genomic DNA
2% (v/v) Di-, Tri-, Tetra-, Penta-, Hexa-, Octa-, or Dodeca-Ethylene glycol

### 40 mM NaCl, 10 mM KCl, 5 mM MgSO₄

### 20 mM Tris/HCl pH 8.8

0.5 mM dNTP mix, 5 mM dATP
400 ng Tte Helicase, 75 ng Et SSB, 80 U modified Gst Pol. LF (all obtained from Biohelix)

The components were pipetted into the reaction tube on ice and tubes have been mixed well. The tHDA reaction was started by putting the reaction tubes into the pre-heated PCR cycler (Biorad CFX 96). The fluorescence was measured over 40 min in 1 min intervals. All measurements were performed in duplicate.

TABLE 9
Average Ct values of tHDA reactions with primer/probe combinations
for the detection of C. trachomatis in the presence of
2% 2-, 3-, 4-, 5-, 6-, 8-, 12-Ethylene glycol.

| | chain length of Ethylene glycol | | | | | | |
|---|---|---|---|---|---|---|---|
| | 2 | 3 | 4 | 5 | 6 | 8 | 12 |
| Ct- | 16.38 | 18.02 | 17.61 | 16.91 | 18.17 | 18.75 | 16.26 |
| average | | | | | | | |

### Example 9

### Effect of Different Ethylene Glycols on tHDA Reactions—Amplification of N. Gonorrhoeae DNA

The components used had the following concentrations in the final reaction volume:

160 nM forward primer (specific for N. gonorrhoeae)
480 nM reverse primer (specific for N. gonorrhoeae)
60 nM probe (specific for N. gonorrhoeae)
100 cp N. gonorrhoeae genomic DNA
2% (v/v) Di-, Tri-, Tetra-, Penta-, Hexa-, Octa-, or Dodeca-Ethylene glycol

### 40 mM NaCl, 10 mM KCl, 5 mM MgSO₄

### 20 mM Tris/HCl pH 8.8

0.5 mM dNTP mix, 5 mM dATP
400 ng Tte Helicase, 75 ng Et SSB, 80 U modified Gst Pol. LF (all obtained from Biohelix)

The components were pipetted into the reaction tube on ice and tubes have been mixed well. The tHDA reaction was started by putting the reaction tubes into the pre-heated PCR cycler (Biorad CFX 96). The fluorescence was measured over 40 min in 1 min intervals. All measurements were performed in duplicate.

TABLE 10
Average Ct values of tHDA reactions with primer/probe combinations
for the detection of N. gonorrhoeae in the presence of
2% 2-, 3-, 4-, 5-, 6-, 8-, 12-Ethylene glycol.

| | chain length of Ethylene glycol | | | | | | |
|---|---|---|---|---|---|---|---|
| | 2 | 3 | 4 | 5 | 6 | 8 | 12 |
| Ct- | 19.77 | 20.77 | 23.02 | 20.67 | 20.89 | 18.93 | 17.16 |
| average | | | | | | | |

### FIGURE CAPTIONS

FIG. 1: Tetraethylene glycol, TEG.

FIG. 2: Time dependent fluorescence of tHDA reactions, during the amplification of a N. gonorhoeae specific DNA sequence, in the presence of 0 to 5% (v/v) ethanol.

FIG. 3: Time dependent fluorescence of tHDA reactions, during the amplification of a N. gonorhoeae specific DNA sequence, in the presence of 0 to 5% (v/v) tetraethylene glycol.

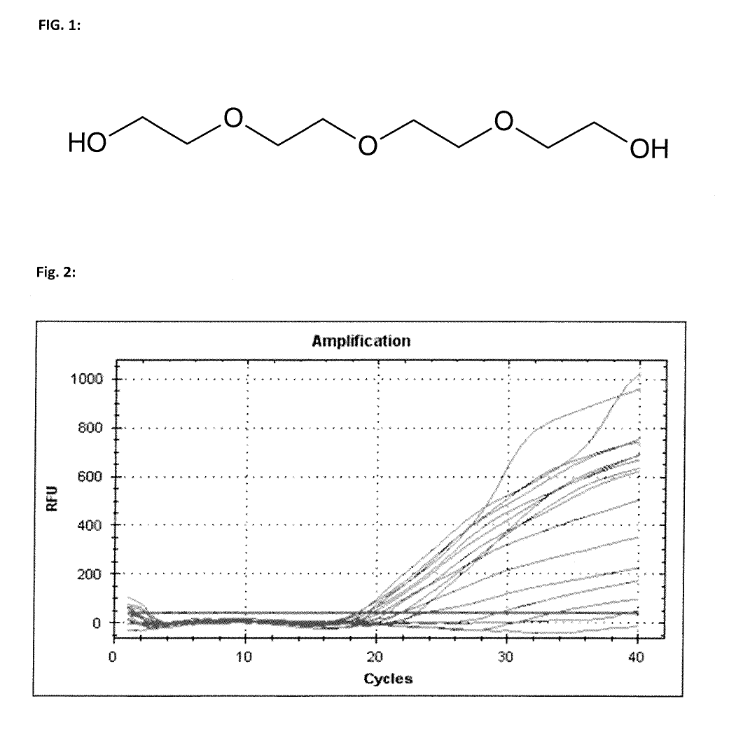

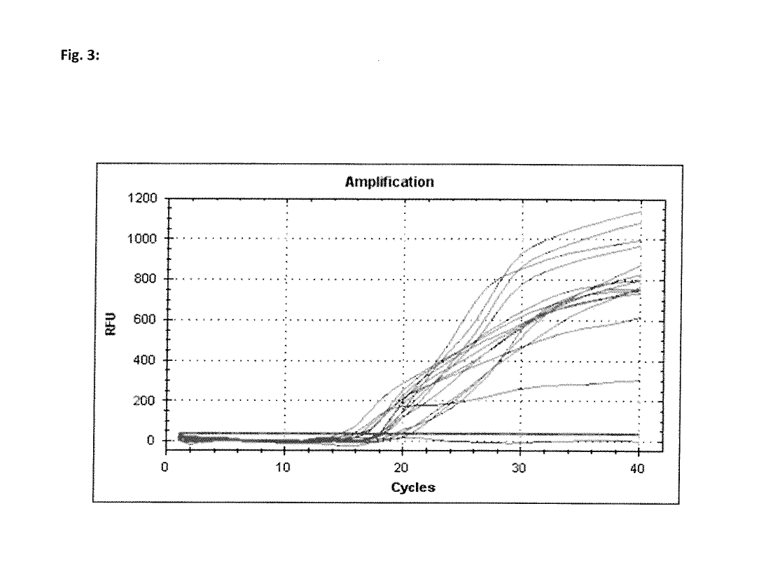

1. A method of performing a helicase dependent amplification (HDA) of one or more nucleic acids templates comprising:
(a) combining in a reaction mixture one or more template nucleic acids; one or more forward and reverse HDA primer, a helicase, at least one DNA polymerase, a buffer and deoxynucleotide triphosphates (dNTPs); and
(b) wherein the reaction comprises a linear polyethylene glycol (PEG) with the following formula: 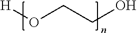
wherein n is 2-50 and the concentration of the PEG is 5% (volume percent) or lower.

2. The method of claim 1, wherein n is selected from the group comprising 2, 3, 4, 5, 6, 7, 8, 9, 10, 11 and 12.

3. The method of claim 1, wherein the polyethylene glycol is present at between 0.1% and 5% (volume percent).

4. The method of claim 1, wherein the polyethylene glycol is present at between 2% and 5% (volume percent).

5. The method of claim 1, wherein the helicase in the reaction is selected form the group of T7 Gp4 helicase, DnaB helicase, Rho helicase, UvrD helicase, PcrA, Rep and NS3 RNA helicase.

6. The method of claim 1, wherein the HDA reaction is multiplex HDA or tHDA.

7. The method of claim 1, wherein the HDA reaction is singlepiex HDA or tHDA.

8. A method according to claim 1, wherein the nucleic acid template was purified using a chaotropic salt/silica method, wherein the purification buffer comprises a linear polyethylene glycol with the formula 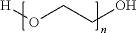
wherein n is between 2 to 50 and the concentration of the PEG is 5% (volume percent) or lower.

9. A method for the preparation of a kit for performing an HDA reaction according to claim 1 comprising the steps of:
providing a helicase and a DNA polymerase;
providing at least one linear polyethylene glycol with the formula 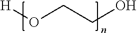
wherein n is between 2 to 50, in such a way, that polyethylene glycol is present at 5% (volume percent) or less; and
combining the provided components in a kit, each in separate form or as premixed components, optionally including instructions, ready for distribution.

10. A kit for performing an HDA reaction according to claim 1, comprising at least a helicase and a DNA-polymerase and at least one polyethylene glycol.

11. A kit according to claim 10, wherein the at least one polyethylene glycol has the formula 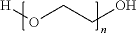
wherein n is between 2 to 50.

12. A kit according to claim 10, wherein the kit is provided in such a way that polyethylene glycol is present at between 0.1% and 5% (volume percent) in the final reaction volume.

13. A kit according to claim 10, additionally comprising one or more reagents for nucleic acid sample preparation, using a chaotropic salt/silica method, wherein the purification buffer comprises TEG.

14. (canceled)

15. The method of claim 1, wherein the HDA is a thermophilic helicase dependent amplification (tHDA).

16. The method of claim 9, further providing a buffer in which both the helicase and DNA polymerase show activity.

17. The method of claim 9, wherein the polyethylene glycol is present at between 0.1% and 5% (volume percent), or between 2% and 5% (volume percent) in the final reaction volume.

18. The method of claim 9, wherein n is 2, 3, 4, 5, 6, 7, 8, 9, 10, 11 or 12.

19. The method of claim 9 further comprising providing deoxynucleotide triphosphates.

20. The kit of claim 10, wherein the HDA is a thermophilic helicase dependent amplification (tHDA).

21. The kit of claim 11, wherein n is 2, 3, 4, 5, 6, 7, 8, 9, 10, 11 or 12.
